(19) United States
(12) Patent Application Publication
(10) Pub. No.: US 2020/0197921 A1
(43) Pub. Date: Jun. 25, 2020

(54) PRECIPITATION CATALYST FOR THE HYDROGENATION OF ETHYL ACETATE CONTAINING COPPER ON ZIRCONIA

(71) Applicant: BASF SE, Ludwigshafen am Rhein (DE)

(72) Inventors: Sabine BORCHERS, Ludwigshafen am Rhein (DE); Marie Katrin SCHROETER, Ludwigshafen am Rhein (DE); Martin MUHLER, Bochum (DE); Katharina TOELLE, Bochum (DE); Sabrina POLIERER, Bochum (DE); Sven ANKE, Bochum (DE); Julian SCHITTKOWSKI, Bochum (DE)

(21) Appl. No.: 16/612,499

(22) PCT Filed: May 9, 2018
(86) PCT No.: PCT/EP2018/062133

§ 371 Date: Nov. 11, 2019

(30) Foreign Application Priority Data
May 12, 2017 (EP) 17170777.1

(51) Int. Cl. B01J 37/03; B01J 6/00; B01J 37/02; B01J 37/06; B01J 21/06; B01J 23/72; B01J 35/10; C07C 31/08

(52) U.S. Cl. CPC B01J 37/031; B01J 6/001; B01J 37/0236; B01J 37/06; B01J 21/066; B01J 2231/641; B01J 35/1066; B01J 35/1019; B01J 35/1061; C07C 31/08; B01J 23/72

(57) ABSTRACT

A process for the preparation of a copper/zirconia-catalyst for the hydrogenation of ethyl acetate to ethanol comprising the steps: a) preparation of an aqueous solution of water-soluble copper and zirconium salts; b) precipitation of a solid from this solution by addition of a basic precipitating agent, and optionally aging of the solid; c) separation and washing of the solid; d) drying of the solid; e) calcination of the solid; characterized in that the precipitation of the solid in step b) is carried out at a pH in the range of from 7 to 7,5, and the basic precipitation agent contains a mixture of Na₂CO₃ and NaOH.

The present invention relates to a process for the preparation of a copper/zirconia-catalyst for the hydrogenation of ethyl acetate to ethanol, the catalyst obtained thereby and the use of the catalyst.

Zirconia-supported catalysts are known to be active and selective for methanol synthesis.

R. A. Koeppel et. al., Applied Catalysis A: General, 84 (1992), pp. 77-102 disclose the preparation of a copper/zirconia-catalyst for the synthesis of methanol from carbon dioxide by co-precipitation from aqueous solutions of copper nitrate and zirconyl nitrate with ammonium carbonate at pH 8.0 and 368 K. The precipitate is aged, filtered and finally calcined at 623 K. In another experiment, copper acetate and zirconyl nitrate was co-precipitated with sodium hydroxide at pH 7 and 368 K, and the gelatinous precipitate treated in the same way. The catalyst was used for the hydrogenation of carbon dioxide to methanol.

Compositions comprising copper oxide and zircon oxide are also known as absorption compositions for the removal of carbon monoxide from material streams.

WO 2007/147783 discloses the preparation of an absorption composition by co-precipitation from a copper and zirconium nitrate solution with a 20% by weight soda solution at pH 6.5 and 70° C. The precipitate is aged at 70° C., washed with water, dried and calcined at 300° C. The tableted composition is reduced in H2/N2 and partly re-oxidized with 0.6 vol.-% O₂ in nitrogen. The composition is used for the removal of CO from a CO containing propylene streams.

Amol M. Hengne, Chandrashekhar V. Rode, Green Chem. 14, (2012), 1064 teach that nano-composites of Cu/ZrO₂ can be used advantageously for the hydrogenation of levulinic acid and its ester to γ-valerolactone. Carbonyl hydrogenations are widely used in industry to produce alcohols from esters or from ketones or aldehydes. The products of these processes may be used in petrochemical processes (e.g. hydrogenation of maleic anhydride and its derivatives), in food processes (e.g. hydrogenation of fats & oils) or in fine chemicals processes (e.g. aroma chemicals production).

U.S. Pat. No. 5,198,592 describes silica- and chromia-supported copper catalysts for the hydrogenation of methyl and ethyl acetate.

Y. Zhu, X. Shi, Bull. Korean Chem. Soc. 35 (1), (2014), 141; O. Kazu, A. Kiyotaka, I. Yasuhiro, J. Phys. Chem. 101, (1997), 9984; and B. Zhang, L. Lin, J. Zhuang, Y. Liu, L. Peng, L. Jiang, Molecules 15, (2010), 5139 describe ethyl acetate hydrogenation on bimetallic copper zinc catalysts supported on silica. The catalysts were prepared by co-precipitation of the corresponding metal hydroxides on silica.

A survey of catalytic hydrogenations of esters to alcohols was published by T. Turek, D. L. Trimm, N. W. Cant, Catal. Rev. 36 (4), (1994), 645. Ester hydrogenations can be performed on different catalytically active metal catalysts. However, copper-based catalysts are described in this paper to be more selective towards alcohols than other hydrogenation catalysts.

Based on the finding that copper is advantageous for ester hydrogenation many different ester hydrogenation processes have been described in the patent literature, yielding alcohols of industrial importance and commercial relevance.

U.S. Pat. No. 1,605,093 describes the hydrogenation of esters, in particular of methyl formate, to the corresponding alcohols using partially reduced compounds of copper oxide and or copper hydroxide. However, using bulk compounds of copper turned out to be disadvantageous because the active copper species loses surface area, thereby deactivating the catalyst slowly and steadily.

One solution for this problem was given by U.S. Pat. No. 6,207,865 that uses Raney copper catalysts for the hydrogenation of esters to alcohols. Raney copper catalysts have the highest possible copper content because they essentially consist of metallic copper only. However, non-stabilized Raney copper catalysts are very prone to sintering even at very mild reaction conditions, thereby losing activity very quickly.

Raney copper catalysts can be stabilized against sintering and the resulting loss in catalytic activity by the presence of other oxides such as zinc oxide. J. R. Mellor, N. J. Coville, A. C. Sofianos, R. G. Copperthwaite, Appl. Catal. A: General 164, (1997), 171 and 185 describe stable Raney copper catalysts for the water-gas shift reaction that contain zinc oxide crystallites, thereby becoming more stable than bare Raney copper.

However, initial activity and longer term stability are insufficient when using Raney copper catalysts. Hence, authors have described copper catalysts that contain copper on solid carriers, mostly on oxide materials.

U.S. Pat. No. 3,213,145 teaches a process for hydrogenating esters of aromatic monocarboxylic acids to aryl-substituted methanols. The process uses copper catalysts supported on alumina. Typically, alumina-based catalysts have the disadvantage that the carrier oxide is chemically disintegrated by the presence of traces of free carboxylic acids.

A solution to this problem was described by H. Adkins, E. E. Burgoyne, H. J. Schneider, J. Am. Chem. Soc. 72 (6), (1950), 2626, using chromium oxide-based copper catalysts. U.S. Pat. No. 2,091,800 teaches a method for producing copper chromite catalysts for the hydrogenation of various carboxylic ester compounds. Copper chromite has proven to be a very stable catalyst for many different hydrogenation reactions on carboxylic esters. An update on the use of this catalyst type was given by R. Prasad, P. Singh, B. Chem. React. Eng. & Catal. 6 (2), (2011), 63.

Chromium oxide as a support for such catalysts has shown to be very resistant to the attack by carboxylic acids present in the reaction mixture of such hydrogenations.

However, copper chromite catalysts have come under much scrutiny in recent years due to the hazardous potential of the chromium contained in the catalyst.

Also, pore structures of copper chromite catalysts are often coarse and, hence, do not allow for a sufficiently high internal surface area that would allow the active copper phase to be dispersed on finely enough for a high catalytic activity. This limits the maximum accessible activity of such copper chromite catalysts.

The task was to develop a highly active and stable copper catalyst for the hydrogenation of ester compounds. The catalyst should be supported on an environmentally friendly oxide support material.

Ethyl acetate is typically hydrogenated to ethanol in various industrial plant configurations. U.S. Pat. No. 8,710,279 teaches a process that converts ethyl acetate formed in the hydrogenation of acetic acid to ethanol via hydrogenolysis yielding additional ethanol product. The authors claim to use copper or group VIII metal catalysts for their process.

It is an object of the present invention to provide an active and selective catalyst for the hydrogenation of ethyl acetate to ethanol. Ethanol is yielded in this process, which can be converted into e.g. ethylene, a valuable petrochemical raw material. As a raw material, ethyl acetate can be taken from various sources, including product streams from biotechnological fermentation routes, as described in K. Nordstrom, J. I. Brewing 67 (2), (2013), 173.

The object is solved by a process for the preparation of a copper/zirconia-catalyst for the hydrogenation of ethyl acetate comprising the steps
a) preparation of an aqueous solution of water-soluble copper and zirconium salts;
b) precipitation of a solid from this solution by addition of a basic precipitating agent and optionally aging of the precipitated solid;
c) separation and washing of the solid;
d) drying of the solid;
e) calcination of the solid;

characterized in that the precipitation of the solid in step b) is carried out at a pH in the range of from 7 to 7.5, and the precipitation agent contains a mixture of Na₂CO₃ and NaOH.

The process may further comprise the additional steps:
f) shaping the solid obtained from step e) to give shaped bodies; or
g) modifying the solid obtained from step c), d) or e) to give powders of modified particles; and
h) optionally forming shaped bodies from the powders obtained in step g);
i) optionally further calcination of the shaped bodies or powders obtained from step f), g) or h);

wherein the shaping step f) can also be carried out between the drying step d) and the calcination step e).

In the first process step, a solution of copper salts and zirconium salts in water is prepared. The preferred copper salt is copper nitrate Cu(NO₃)₂, the preferred zirconium salt is zirconyl nitrate ZrO(NO₃)₂. Another possible zirconium salt is zirconyl chloride ZrOCl₂. The quantitative ratio of the salts in the solution is calculated and set stoichiometrically according to the desired final catalyst composition. In general, the atomic ratio of Cu:Zr in the aqueous solution is in the range of from 3:1 to 1:3, preferably in the range of from 1:3 to 2:5, and is most preferred 1:3.

From this solution, in step b) a solid is precipitated as precursor of the catalyst composition. This is done by increasing the pH of the solution by adding a base as precipitating agent. According to the invention, the basic precipitating agent is a mixed soda/sodium hydroxide solution. The solid is precipitated at a pH of from 7.0 to 7.5, preferably from 7 to 7.3 and most preferably 7. Preferably, the pH is kept constant to ±0.1 during precipitation.

In general, the overall content of soda and sodium hydroxide in the aqueous solution of the precipitating agent is in the range of from 5 to 30% by weight. The ratio of Na₂CO₃:NaOH is in the range of from 2:1 to 10:1, preferably form 5:1 to 10:1, e.g. 7:1. In a preferred embodiment, an aqueous solution containing 25% by weight of Na₂CO₃:NaOH in a ratio of 7:1 is used as the precipitating agent.

A ZrO₂ gel matrix, with copper embedded as CuCO₃, is obtained as precipitate.

It has been surprisingly found that a very active and selective catalyst can be obtained if co-precipitation is carried out at pH 7-7.5 using this particular precipitating agent. The catalyst of the invention is much more active and selective towards the formation of ethanol as compared to a catalyst precipitated with a different basic precipitating agent, or precipitated at a higher pH.

The precipitated solid can be aged at the same pH as maintained during precipitation, usually by further stirring for 1 min to 2 h.

The resultant solid precipitate, before the drying in step c), is generally separated off from the supernatant solution, for instance by filtering or decanting, and washed free from soluble components such as sodium nitrate with water.

The precipitation product is then, usually before further processing, dried in a drying step d) using conventional drying methods. Generally, a treatment at a slightly elevated temperature, for instance about 80° C., preferably at least 105° C., and also generally at most 160° C., preferably at most 140° C. over a period of 8 to 36 hours, preferably 12 hours to 24 hours, suffices for this.

Subsequently to the drying, the precipitated and dried intermediate product of the adsorption composition is subjected to calcination step e). The calcination temperature employed in this case is generally at least 350° C., preferably at least 400° C., and in a particularly preferred manner at least 450° C., and also generally at most 650° C., preferably at most 600° C., and in a particularly preferred manner at most 550° C. One example of a highly suitable temperature window for this calcination is the range from 470 to 530° C., that is to say 500±30° C. The calcination time is generally at least 20 minutes, preferably at least 40 minutes, and in a particularly preferred manner at least 60 minutes, and also generally at most 12 hours, preferably at most 6 hours, and in a particularly preferred manner at most 4 hours.

After the drying step d) or the calcination step e), the composition or its precursor may be processed in the shaping step f) using conventional shaping processes such as rod extrusion, tableting or palletizing to give shaped bodies such as extruded rods or extrudates, tablets, or pellets, including spherical pellets.

Alternatively, the composition or its precursor may be applied in slurry-phase catalytic reaction processes as a powder. The catalyst powder may be used as obtained from the aforementioned preparation steps a) through e). Alternatively, the product from step e) or the product from one of the steps c) or d) may be modified to a powder of modified particle size and shape by an additional step g) such as e.g. redispersing the powders and spray-drying. For this additional step g) a binder may optionally be mixed with the powder. The powder may also be modified by optionally adding a binder and agglomerating the particles, e.g. by a milling process. A modified powder obtained by such an additional step may be applied in catalytic reaction processes. Alternatively, it may be likewise shaped into macroscopic shaped bodies by a shaping step f) as described above. Powders and shaped bodies obtained from one of the steps f), g) or h) may be further processed by a calcination step i).

After a shaping step f) or h) or a modifying step g), the catalyst composition can be subjected to a further calcination step i). The calcination temperature employed in this case is generally at least 300° C., preferably at least 350° C., and in a particularly preferred manner at least 400° C., in particular at least 450° C., and also generally at most 700° C., preferably at most 650° C., and in a particularly preferred manner at most 600° C., in particular at most 580° C. One example of a highly suitable temperature window for this calcination step is the range from 470 to 550° C., in particular in the range from 500 to 540° C. The calcination time is generally at least 30 minutes, preferably at least 60 minutes, and also generally at most 24 hours, preferably at most 12 hours.

In case the shaping step f) is carried out after the drying step d) but before the calcination step e), only calcination step e) is carried out.

Preferably, the obtained catalyst contains from 18 to 30% by weight of CuO and from 70 to 82 by weight of ZrO₂.

The calcined catalyst is X-ray amorphous without detectable crystallinity. The specific surface area of the X-ray amorphous catalysts is generally in the range from 100 to 160 m²g⁻¹, preferably from 110 to 130 m²g⁻¹. The average pore diameter is generally in the range from 2.3 to 3.6 nm, preferably in the range from 2.9 to 3.3 nm.

The catalyst composition prepared according to the invention can also be deposited on a support. This is performed by conventional impregnation processes or deposition precipitation. A deposition precipitation as is known is a precipitation process in the presence of a support of a support precursor. For carrying out a deposition precipitation, preferably in the above described precipitation process, a support or support precursor is added to the solution produced in step a). If the support is already present in the form of preshaped finished shaped bodies, therefore a pure impregnation process is omitted from shaping step e), otherwise the support is formed during processing the intermediate of the adsorption composition by precipitation, drying, calcination and shaping.

In the production of the composition, use can be made of known auxiliaries, for instance pore forming agents or tableting aids which decompose in the calcination.

The catalyst prepared according to the invention can be made up into any suitable form. The active composition can be used in the form of catalyst shaped bodies, as a monolith or as catalytically active layer applied to a support (metal or ceramic).

The invention also relates to a catalyst for the preparation of a copper/zirconia-catalyst for the hydrogenation of ethyl acetate, obtainable by the inventive process.

The invention also relates to the use of the catalyst for the hydrogenation of ethyl acetate to ethanol after reduction of the catalyst.

Usually, the calcination is carried out under air, and copper is therefore present in the form of CuO in the precursor of the adsorption composition of the invention obtained after calcination. The degree of reduction is then set to the desired degree of reduction by reducing the copper. This is performed by treating the precursor present after calcination with a reducing agent. Any known reducing agent can be used which can reduce copper. The exact reaction conditions to be employed depend on the precursor and its composition and also on the reducing agent used and can readily be determined in a few routine experiments. A preferred process is treating the precursor with hydrogen, usually by passing over a hydrogen-comprising gas, preferably a hydrogen/nitrogen mixture, at elevated temperature.

Complete reduction of the precursor of the adsorption composition proceeds via reduction of the copper present in the adsorption composition to copper metal. This can proceed in principle via any reducing agent which can reduce copper from oxidation states I or II to oxidation state 0. This can proceed using hydrogen by passing a hydrogen-comprising gas over the precursor.

The temperature to be employed in this case is generally at least 100° C., preferably at least 110° C., and in a particularly preferred manner at least 120° C., and also generally at most 380° C. is reached, preferably at most 360° C., and in a particularly preferred manner at most 340° C. A suitable temperature is, for example, approximately 130° C. The reduction is exothermic. The amount of recirculated reducing agent must be set in such a manner that the temperature window selected is not left. The course of the activation can be followed on the basis of the temperature measured on the bed of the adsorption medium (“temperature-programmed reduction, TPR”).

A preferred method for reducing the precursor of the composition is, subsequently to drying carried out under a nitrogen stream, to set the desired reduction temperature and to admix to the nitrogen stream a small amount of hydrogen. A suitable gas mixture comprises at the start, for example at least 0.1% by volume hydrogen in nitrogen, preferably at least 0.5% by volume and in a particularly preferred manner at least 1% by volume, and also at most 10% by volume, preferably at most 8% by volume, and in a particularly preferred manner at most 5% by volume. A suitable value is, for example, 2% by volume. This initial concentration is either retained or elevated in order to attain and maintain the desired temperature window. The reduction is complete when, despite constant or increasing level of the reducing agent, the temperature in the bed of the composition falls. A typical reduction time is generally at least 1 hour, preferably at least 5 hours, and in a particularly preferred manner at least 10 hours, and also generally at most 50 hours, preferably at most 30 hours, and in a particularly preferred manner at most 20 hours.

In the reduced state, copper nanoparticles are embedded in an amorphous ZrO₂ matrix, and thus well stabilized against sintering.

The hydrogenation of ethyl acetate to ethanol is in general carried out at a hydrogen pressure of from 1 to 200 bar and a temperature of from 200 to 300° C. The ratio of EtOAc to H₂ should be in a range of 1:10 to 1:99, preferably at least 1:40 to 1:80, and in a particularly preferred manner at most 1:70. A suitable value is, for example, 1:68.

### EXAMPLES

Catalyst Preparation

### Example 1 and Comparative Examples C1-C3

Four Cu/ZrO₂ catalysts with identical copper loading were synthesized by co-precipitation in a batch process varying the precipitating agent and the pH. Initially, ZrO(NO₃)2×3 H₂O and Cu(NO₃)₂×3 H₂O were dissolved in water. For a 18.3 wt % CuO/ZrO₂, 6.0317 g ZrO(NO₃)₂. 3H₂O and 1.6677 g Cu(NO₃)2.3 H₂O were dissolved together in 45 mL water. During the coprecipitation, the metal nitrates were pumped (ICP pump, ISMATEC) continuously into the precipitation reactor filled with 200 mL HPLC water. Simultaneously, the pH was kept constant at pH 10.5 or pH 7 with 25 wt % NaOH (OH-10, OH-7) or 7:1 Na₂CO₃/NaOH (saturated solution/25 wt %) (CO₃-10, CO₃-7) as precipitating agents. The addition of the precipitating agent was controlled by an autotitrator (Titroline alpha, Schott) connected with a pH electrode (Schott) located in the precipitation reactor. After co-precipitation, the solution containing the precursor was aged for 15 min. Subsequently, the precursor was filtered, washed with 0.75 L water until the nitrate anions were removed, and dried at 378 K for 18 h. Finally, the dried precursor was calcined in synthetic air at 763 K for 3 h with a heating rate of 2 K min⁻¹. After calcination, the catalyst was characterized by XRD, N₂ physisorption, TPR, and N₂O-RFC and tested after reduction in the gas-phase hydrogenation of ethyl acetate.

TABLE 1
List of precipitation agents, pH and catalyst samples

| Example | Precipitation agent | pH | Label |
|---|---|---|---|
| C1 | NaOH | 10 | OH-10 |
| C2 | NaOH | 7 | OH-7 |
| C3 | Na₂CO₃/NaOH | 10 | CO₃-10 |
| 1 | Na₂CO₃/NaOH | 7 | CO₃-7 |

Characterisation:

N₂ physisorption measurements were performed at 77 K with 200 mg calcined catalyst using a BELSORP-max volumetric sorption set-up (BEL Japan, Inc.). Before the measurement, the catalyst was heated to 473 K for 2 h under vacuum to remove surface water. X-ray powder diffraction measurements were carried out to characterize the phase composition of the calcined catalysts and the catalysts after hydrogenation of ethyl acetate. Diffraction patterns were recorded in reflection geometry with an Empyrean Theta-Theta diffractometer (Panalytical, Almelo) equipped with a copper tube, 0.25° divergent slit, 0.5° antiscatter slit, 7.5 mm high antiscatter slit, 0.04 rad incident and diffracted beam soller slits, as well as a position sensitive PIXcel-1d detector. For qualitative phase analysis, the catalyst was scanned in the range of 5° -80° 2θ with a step width of 0.0131°. Afterwards, the qualitative phase analysis was processed by the ICDD powder diffraction file (PDF2) in conjunction with the HighScore Plus software (Panalytical, Almelo). Additionally, the Cu particle size of the catalysts after hydrogenation was calculated using the Scherrer equation with β=0.053. Temperature-programmed reduction (TPR) and N₂O reactive frontal chromatography (RFC) experiments were performed in a flow-set-up with 100 mg catalyst loaded in a glass-lined stainless steel U-tube reactor. The calcined catalyst was heated up to 513 K with 1 K min⁻¹ in a 84 mL min⁻¹ flow of 5% H₂/Ar. Simultaneously, the hydrogen consumption was measured with a thermal conductivity detector. After cooling to room temperature, N₂O-RFC was carried out with a 10 mL min⁻¹ flow of 1% N₂O/He. The hydrogenation of ethyl acetate was performed in a flow-set-up described below. 100 mg catalyst was loaded in a glass-lined stainless steel U-tube reactor. The catalyst was reduced in a 10 mL min⁻¹ flow of 2% H₂/He with a temperature plateau at 398 K. The maximum temperature during reduction was set to 573 K. For the hydrogenation of ethyl acetate, the reduced catalyst was heated up from room temperature to 513 K with 0.5 Kmin⁻¹ in a gas flow of 50 mL min⁻¹ containing 1% ethyl acetate and 68% hydrogen. After holding the maximum temperature for 1 h, the temperature was reduced to room temperature with 0.5 mL min⁻¹. All gases were analyzed by a calibrated quadrupole mass spectrometer (QMS, GAM422, Balzers).

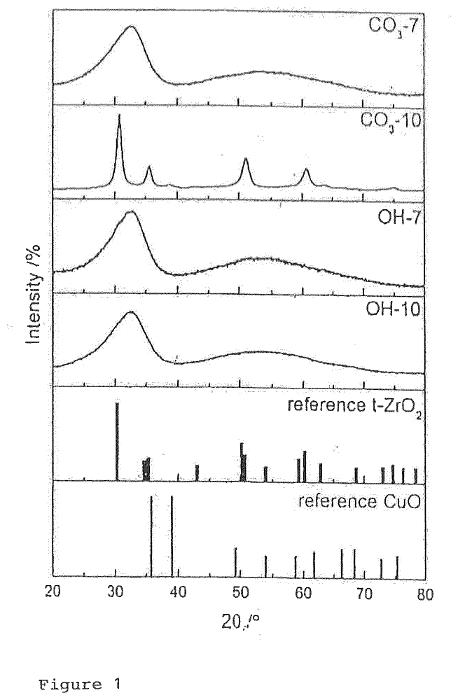

The XRD diffraction patterns of the calcined CuO/ZrO₂ catalyst precursors are shown in FIG. 1 together with the reference patterns of t-ZrO₂ and CuO. In the XRD patterns of the CuO/ZrO₂ catalysts OH-10, OH-7, and CO₃-7, two very broad peaks in the range of 20° to 40° and 45° to 65° 2θ were observed suggesting the presence of essentials amorphous t-ZrO₂. In addition to the two very broad peaks of OH-7, the main reflection for t-ZrO₂ was detected weakly. Therefore, OH-10 and CO₃-7 are essentially X-ray amorphous without detectable crystallinity, and OH-7 is mainly amorphous with some crystallized t-ZrO_{2.} In contrast, for CO₃-10 the main reflections of t-ZrO₂ were clearly observed at 30.2°, 35.3°, 50.3° , and 60.2° 2θ. Furthermore, for CO₃-10 the main reflections of CuO were identified at 35.6° and 38.8° 2θ. Consequently, crystalline t-ZrO₂ and CuO nanoparticles are present in the calcined catalyst precursor CO₃-10, whereas the other precursors are all X-ray amorphous.

The determined surface areas (BET method), average pore diameters (BJH method), and average pore volumes derived from N₂ physisorption results are summarized in Table 2. These results have to be interpreted carefully due to the complex pore network. The specific surface areas of the X-ray amorphous catalysts OH-10, OH-7, and CO₃-7 are in the range from 111 to 119 m²g⁻¹. The average pore diameters are similar amounting to about 2.7 nm. In comparison to the X-ray amorphous catalysts, the specific surface area of the crystalline catalyst precursor CO₃-10 is smaller by 2/3, and the average pore diameter of 6.7 nm is about twice the size. The average pore volumes are comparable for all four samples.

TABLE 2
Specific surface areas, average pore diameters and average pore
volumes of the calcined CuO/ZrO₂ precursors precipitated with
NaOH and Na₂CO₃/NaOH at pH 10.5 and 7

| | | | | Average pore |
|---|---|---|---|---|
| | | Specific BET | Average pore | volume/ |
| Example | Sample | surface/m²g⁻¹ | diameter/nm | cm³g⁻¹ |
| C1 | OH-10 | 119 | 2.7 | 0.08 |
| C2 | OH-7 | 111 | 2.4 | 0.07 |
| C3 | CO₃-10 | 38 | 6.7 | 0.06 |
| 1 | CO₃-7 | 114 | 3.1 | 0.09 |

The degrees of reduction derived from TPR experiments were approximately 100% and are summarized in Table 3 together with the reduction temperatures and specific copper surface areas derived from N₂O reactive frontal chromatography (RFC) performed with the reduced Cu/ZrO₂ catalysts. Obviously, CuO was fully reduced to metallic Cu. The reduction temperatures of these catalysts were shifted depending on the chemical nature of the oxidic Cu precursor. The lowest reduction temperature of 399 K was found for CO₃-7 followed by the reduction temperature of 407 K for OH-10. For OH-7, a reduction temperature of 415 K was detected, whereas the reduction profile for CO₃-10 was found to extend over a broad temperature range with a peak at 429 K. Thus, the reduction temperature was 30 K higher than that of CO₃-7. The reduction temperature of pure CuO which is not shown in this work is estimated to 526 K. The specific Cu surface areas were in the range of 1.4 m²g_{cat}⁻¹ to 5.2 m²g_{cat}⁻¹.

TABLE 3
TPR and N₂O-RFC results of CuO/ZrO₂ catalysts precipitated
with NaOH and Na₂CO₃/NaOH at pH 10.5 and 7

| | | Degree of | Reduction | Specific Cu surface |
|---|---|---|---|---|
| Example | Sample | reduction/% | temperature/K | area/m²g_{cat}⁻¹ |
| C1 | OH-10 | 96 | 407 | 1.4 |
| C2 | OH-7 | 100 | 413 | 2.1 |
| C3 | CO₃-10 | 100 | 429 | 4.0 |
| Ex. 1 | CO₃-7 | 100 | 399 | 5.2 |

Hydrogenation

### Example 2 and Comparative Examples C4-C6

Microreactor Setup:

The hydrogenation of ethyl acetate and the IR studies were performed in a stainless steel microreactor flow setup with coupled FT-IR.

The setup has three major sections: gas supplies, reaction chamber, and on-line analysis using a quadrupole mass spectrometer (QMS). The piping is made of stainless steel, which is heated to 383 K to prevent condensation. The gas flows are regulated by calibrated mass flow controllers (MFCs) and a combination of pneumatic and manual Valco valves. The flow rates, the pneumatic Valco valves, as well as the reactor temperature and the heating rate are controlled by LabView software. Through LabView, there is the possibility to program sequences. Therefore, the pre-treatment and the hydrogenation is performed equally for each catalyst. The use of a saturator allows the vaporisation of a liquid like ethyl acetate, ethanol, and acetaldehyde into the gas phase. The saturator is kept at 273 K by a Lauda Ecoline RE112 cryostat with ethylene glycol as the cooling fluid. The QMS was calibrated using the following gases: 3.2146% ethyl acetate in He (99.9999%), 1.5710% ethanol in He (99.9999%), 43.8451% acetaldehyde in He (99.9999%), 10% CO (99.997%) in He (99.9999%), 4% CO₂ (99.9995%) with 1% Ar (99.9999%) in He (99.9999%), and 2% H₂ in He (both 99.9999%). A glass-lined U-shaped stainless steel tube with an inner diameter of 4 mL is used as a reactor (MR) that is placed in an aluminium block oven for heating with a maximum temperature of 850 K and a maximum heating rate of 20 K. 100 mg of catalyst with a consistent particle size of 250-355 μm sieve fraction is loaded into the reactor between two glass wool plugs. The temperature during the pre-treatment and reaction is measured by a thermocouple that is inserted directly into the catalyst bed. The thermocouple for the temperature regulation is set in the aluminium block oven. The temperature regulation is performed by a Eurotherm controller and LabView. The setup is coupled with a Nicolet Nexus FT-IR spectrometer, which contains a nitrogen-cooled mercury cadmium telluride (MCT) detector and a DRIFTS cell with ZnSe windows. The temperature of the cell and the heating rate is controlled by Eurotherm and LabView. The FT-IR data is collected and processed through the Omnic software. The reaction chamber also contains a high-pressure unit

(HPU) and connections for attachment of a portable reactor (PR) to the setup; which were not utilized in this study. The quadrupole mass spectrometer (QMS, Balzers GAM422) for the time-resolved quantitative on-line gas analysis is composed of a crossbeam ion source and a secondary electron multiplier (SEM). The use of the QMS allows for simultaneous detection of all reactants and products by using the Quadstar software for calculating conversion and yields.

Hydrogenation: The hydrogenation of ethyl acetate was performed in the microreactor flow set-up described above. 100 mg catalyst was loaded in a glass-lined stainless steel U-tube reactor. The catalyst was gently reduced in 10 ml\per of a flow of 2% H₂/He with a temperature plateau at 398 K to avoid sintering. The maximum temperature during reduction was set to 573 K. For the hydrogenation of ethyl acetate, the reduced catalyst was heated from room temperature to 513 K with 0.5 K/min in a gas flow of 50 ml/min containing 1% ethyl acetate and 68% hydrogen. After keeping the maximum temperature for 1 h, the catalyst was cooled to room temperature with 0.5 K/min.

The catalytic activity of the Cu/ZrO₂ catalysts was assessed in the hydrogenation of ethyl acetate (EtAc) to ethanol (EtOH). The degrees of ethyl acetate conversion and the yields of ethanol at a reaction temperature of 513 K are summarized in Table 4. The highest conversion of ethyl acetate was observed for CO₃-7 with 50% and an ethanol yield of 41%. Furthermore, also OH-10 was highly active with a conversion of 42% and 35% ethanol yield. Only half the conversion of OH-10 was achieved with OH-7. The lowest activity was obtained with CO₃-10 with a conversion of 18% and a yield of 15%.

TABLE 4
Degrees of conversion of ethyl acetate and yields of ethanol at
513K and the calculated Cu particle sizes derived from the XRD
patterns after reaction

| | | | | Cu particle |
|---|---|---|---|---|
| Example | Catalyst | X_{EtAc}/% | Y_{EtOH}/% | size/nm |
| C4 | OH-10 | 42 | 35 | 5 |
| C6 | OH-7 | 20 | 16 | 4 |
| C6 | CO₃-10 | 18 | 14 | 9 |
| 2 | CO₃-7 | 50 | 41 | 5 |

1-14. (canceled)

15. A process for the preparation of a copper/zirconia-catalyst for the hydrogenation of ethyl acetate to ethanol comprising the steps:
a) preparation of an aqueous solution of water-soluble copper arid zirconium salts;
b) precipitation of a solid from this solution by addition of a basic precipitating agent, and optionally aging of the solid;
c) separation and washing of the solid;
d) drying of the solid;
e) calcination of the solid;
characterized in that the precipitation of the solid in step b) is carried out at a pH in the range of from 7 to 7.5, and the basic precipitation agent contains a mixture of Na₂CO₃ and NaOH.

16. The process of claim 15, further comprising the steps:
f) shaping the solid obtained from step e) to give shaped bodies; or
g) modifying the solid obtained from step c), d) or e) to give powders of modified particles; and
h) optionally forming shaped bodies from the powders obtained in step g);
optionally further calcination of the shaped bodies or powders obtained from step f), g) or h);
wherein the shaping step f) can also be carried out between drying step d) and calcination step e).

17. The process according to claim 15, wherein the copper salt is copper nitrate Cu(NO₃)₂ and the zirconium salt is zirconyl nitrate ZrO(NO₃)₂.

18. The process according to claim 15, wherein the atomic ratio of Cu:Zr in the aqueous solution is in the range of from 1:3 to 2:5.

19. The process according to claim 15, wherein the ratio of Na₂CO₃:NaOH in the precipitation agent is in the range of from 2:1 to 10:1.

20. The process according to claim 15, wherein the precipitation agent is employed as an aqueous solution containing from 5 to 30% by weight of Na₂CO₃ and NaOH.

21. The process according to claim 15, wherein in step b) the precipitated solid is aged at a pH in the range of from 7.0 to 7.5.

22. The process according to claim 15, wherein the solid obtained in step c) is dried at a temperature of from 80 to 160° C.

23. The process according to claim 15, wherein the dry solid obtained in step d) is calcined at a temperature of from 350 to 650° C.

24. A catalyst for the preparation of a copper/zirconia-catalyst for the hydrogenation of ethyl acetate, obtainable by the process as claimed in claim 15.

25. The catalyst according to claim 24, containing from 18 to 30% by weight of CuO and from 72 to 80% by weight of ZrO₂.

26. The catalyst according to claim 24, having a specific surface (BET) area in the range from 100 to 130 m2g₋₁ and an average pore diameter (BJH) in the range from 2.6 to 3.6 nm.

27. The catalyst according to claim 24, which is X-ray amorphous.

28. A method of use of the catalyst according to claim 24, wherein ethyl acetate is hydrogenated to ethanol at a hydrogen pressure of from 1 to 200 bar and a temperature from 200 to 300° C. in the pressure of said catalyst.
